(19) United States
(12) Patent Application Publication
(10) Pub. No.: US 2018/0021243 A1
(43) Pub. Date: Jan. 25, 2018

(54) AGENTS FOR TEMPORARILY SHAPING AND SIMULTANEOUSLY LIGHTENING KERATIN FIBERS

(71) Applicant: Henkel AG & Co. KGaA, Duesseldorf (DE)

(72) Inventors: Marc Krippahl, Moenchengladbach (DE); Burkhard Mueller, Duesseldorf (DE); Constanze Neuba, Grevenbroich (DE)

(73) Assignee: Henkel AG & Co. KGaA, Duesseldorf (DE)

(21) Appl. No.: 15/654,139

(22) Filed: Jul. 19, 2017

(30) Foreign Application Priority Data
Jul. 20, 2016 (DE) 102016213268.7

(51) Int. Cl. A61K 8/81; A45D 7/04; A61Q 5/06; A61Q 5/08; A61K 8/22; A61K 8/73

(52) U.S. Cl. CPC A61K 8/8182; A61K 8/22; A61K 8/73; A61Q 5/06; A61Q 5/08; A45D 7/04; A61K 2800/48; A45D 2007/001

(57) ABSTRACT

Cosmetic agents for simultaneously lightening and temporarily shaping keratin fibers include a specific thickener and also at least one oxidant and have a viscosity of from 31,000 to 90,000 mPas. Despite the high viscosity, the agents can be easily removed from the packaging and moreover can be homogeneously applied to the keratin fibers without said agents dripping after application. The agents result in a homogeneous lightening and a good hairstyle hold. A method for lightening and temporarily shaping keratin fibers uses such cosmetic agents.

### FIELD OF THE INVENTION

The present invention generally relates to a cosmetic agent for temporarily shaping and simultaneously lightening keratin fibers. The present invention additionally relates to the use of such cosmetic agents and also to corresponding use methods.

### BACKGROUND OF THE INVENTION

An attractive hairstyle is nowadays generally regarded as an indispensable part of a well-groomed appearance. Based on current fashion trends, hairstyles which are considered to be chic are often those which on many hair types can be formed and/or maintained for a relatively long period of time of up to several days only by using active setting ingredients. Hair treatment agents which serve for temporarily shaping the hair therefore play an important role. Such temporary shaping agents usually contain synthetic polymers and/or waxes as the active setting ingredient.

There is also a demand not only for changing the hairstyle by means of temporary shaping but also for simultaneously lightening the hair color. Many lightening products are available to consumers for lightening hair. Lightening products are nowadays much gentler than before and even make it possible to lighten very dark hair. Blonding agents lighten medium-blonde or light-brown hair by up to eight shades. Specific products for very dark hair ranging from medium-brown to black enable lightening by two to three shades.

So-called “blonding sprays” for spray application are already known in the prior art. Due to the low concentration of oxidants, in particular hydrogen peroxide, the hair can be lightened by around one-quarter of a shade upon each application. Such sprays contain a lightening liquid of low viscosity which is finely distributed on the hair by means of a pump with a spray head. If the user sprays too much lightening liquid, the liquid may drip from the hair due to the low viscosity. Furthermore, a temporary shaping of the keratin fibers is not provided by the use of such sprays.

Accordingly, it is desirable to provide a cosmetic agent for temporarily shaping and lightening keratin fibers, which agent exhibits a lower tendency to drip after application and at the same time leads to good long-term hold, good flexibility and an excellent humidity resistance of the hairstyle. In addition, such a cosmetic agent should be storage-stable and easy to handle, in particular easy to remove from the packaging and easy to distribute on the keratin fibers.

### BRIEF SUMMARY OF THE INVENTION

A first subject matter of the present invention is thus a cosmetic agent for lightening and temporarily shaping keratin fibers, containing, in a cosmetically acceptable carrier,
a) at least one thickener, selected from xanthan gum and/or crosslinked anionic copolymers, including at least one structural unit of formula (I) and at least one structural unit of formula (II)

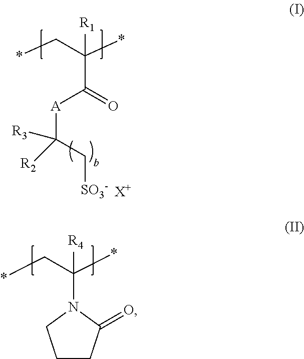

in which
R₁ and R₄ each independently of one another represent hydrogen or a C₁₋₄ alkyl group,
R₂ and R₃ each independently of one another represent a C₁₋₄ alkyl group,
A represents O, NH or S,
b represents integers from 1 to 5,
X⁺ represents a physiologically acceptable cation, and
b) at least one oxidant,
   wherein the cosmetic agent has a viscosity of from 31,000 to 90,000 mPas (Brookfield RV, spindle T96, 4 rpm).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now surprisingly been found that the use of particular thickeners in combination with at least one oxidant leads to cosmetic agents which, due to their high viscosity, do not drip from the hair after application and at the same time enable a temporary shaping of keratin fibers. In addition, said agents can be easily distributed on the keratin fibers due to the high viscosity. The shaping exhibits good long-term hold, good flexibility, and excellent humidity resistance. Moreover, the thickeners used are stable with respect to the oxidants used, so that the agents according to the invention are storage-stable for a long period of time.

By combining a specific thickener with an oxidant, the viscosity of the agents according to the invention can be set so that said agents do not drip after application to the keratin fibers and moreover can be easily distributed in the hair. In this way, an excellent and uniform blonding is achieved even with small amounts of oxidants. In addition, the use of said thickeners also enables a temporary shaping of the keratin fibers. Therefore, with the cosmetic agent according to the invention, both an excellent and uniform blonding and also good long-term hold and good flexibility of the keratin fibers shaped using said agents are achieved.

According to the formulae above and all the formulae below, a chemical bond marked by the symbol “*” represents a free valency of the structural fragment in question. Here, free valency is understood to mean the number of atomic bonds which start from the structural fragment in question at the position marked by the symbol “*.” In the context of the present invention, preferably in each case one atomic bond starts from the positions of the structural fragments marked by the symbol “*” toward further structural fragments.

Keratin fibers will in principle be understood to mean all types of animal hair, for example wool, horsehair, angora hair, furs, feathers, and products or textiles manufactured therefrom. Preferably, however, the keratin fibers are human hair.

Furthermore, in the context of the present invention, the term “thickener” will be understood to mean compounds which are able to bind liquids, in particular water, and increase the viscosity of said liquids. In the context of the present invention, these also include gelling agents which are able to thicken liquids to form compositions having a gel-like consistency or to form gels. According to the invention, gel-like cosmetic agents or gels will be understood to mean shape-stable, easily deformable, disperse systems composed of at least two components, the gelling agent (usually a solid, colloidally dispersed substance comprising long or highly branched compounds) and a liquid (usually water) as the dispersion medium. The gelling agent forms a spatial network in the liquid, wherein the individual gel-forming compounds adhere to one another through primary and/or secondary valencies at different spatial points.

In addition, in the context of the present invention, the term “anionic copolymers” will be understood to mean those polymers which contain at least two different monomers and which in a protic solvent under standard conditions carry at least one structural unit having permanently anionic groups, wherein the anionic groups are fully or partially compensated by counter-ions to maintain electroneutrality. According to the invention, anionic groups include in particular carboxyl groups and sulfonic acid groups.

“Crosslinked copolymers” will be understood to mean those copolymers in which the polymer chains are linked to one another either by a direct covalent bond between the individual polymer chains or by bridging molecule fragments which are each covalently bound to the polymer chains. Crosslinked copolymers in the context of the present invention therefore have a network formed by covalent chemical bonds.

Furthermore, the term “physiologically acceptable cations” will be understood to mean those cations which are non-poisonous and/or non-toxic and/or non-hazardous to the organism, in particular the human and animal organism.

In addition, the term “fatty acid,” as used in the context of the present invention, will be understood to mean aliphatic carboxylic acids which have unbranched or branched carbon residues containing 4 to 40 carbon atoms. The fatty acids used in the context of the present invention may be both naturally occurring and synthetically produced fatty acids. Furthermore, the fatty acids may be monounsaturated or polyunsaturated.

Finally, the term “fatty alcohol” in the context of the present invention will be understood to mean aliphatic, monovalent, primary alcohols which have unbranched or branched hydrocarbon residues containing 4 to 40 carbon atoms. The fatty alcohols used in the context of the invention may also be monounsaturated or polyunsaturated.

Unless indicated otherwise, the specification of the total amount in respect of the components of the cosmetic agent relates to the total amount of active substance of the respective component. Furthermore, unless indicated otherwise, the specification of the total amount in respect of the components of the cosmetic agent relates to the total weight of the cosmetic agent according to the invention.

The cosmetic agents according to the invention contain a cosmetic carrier. According to the invention, the cosmetic carrier is preferably aqueous, alcoholic or aqueous-alcoholic. In the context of the present invention, it is particularly preferred if the cosmetic agents are prepared as thickened solutions in a packaging. It is therefore particularly preferred if the cosmetic agents according to the invention are removed from a packaging, in particular a tube or a pot, and then are applied to the keratin fibers, in particular the hair.

An aqueous carrier in the context of the invention contains at least 30 wt %, in particular at least 50 wt % water, in relation to the total weight of the cosmetic agent.

In the context of the present invention, aqueous-alcoholic carriers are to be understood to mean water-containing compositions containing a C₁-C₄ alcohol in a total amount of from 3 to 90 wt %, in relation to the total weight of the cosmetic agent, in particular ethanol or isopropanol.

The carrier may additionally contain further organic solvents, such as for example methoxybutanol, ethyl diglycol, 1,2-propylene glycol, n-propanol, n-butanol, n-butylene glycol, glycerol, diethylene glycol monoethyl ether, and diethylene glycol mono-n-butyl ether. Preference is given to all water-soluble organic solvents, wherein the solvent is contained in a total amount of from 0.1 to 30 wt %, preferably from 1 to 20 wt %, in particular from 2 to 10 wt %, in relation to the total weight of the cosmetic agent.

As a first essential constituent a), the cosmetic agent according to the invention contains at least one thickener, selected from the group consisting of xanthan and/or specific crosslinked anionic copolymers based on the structural units (I) and (II). In the structural units (I) and (II), the residues R₁ to R₄ may represent a C₁-C₄ alkyl group. Examples of these are methyl, ethyl, propyl, isopropyl, butyl, sec-butyl, isobutyl, and tert-butyl groups.

According to the invention, the crosslinked anionic copolymer preferably contains structural units (I) and (II) in which the residues R₁ and R₄ each represent hydrogen. Preferred embodiments of the present invention are therefore characterized in that the residue R₁ in the structural unit of formula (I) and the residue R₄ in the structural unit of formula (II) each represent hydrogen.

It is furthermore preferred if, in the structural unit of formula (I), the residues R₂ and R₃ each represent a particular alkyl group. It is therefore preferred according to the invention if, in the structural unit of formula (I), the residues R₂ and R₃ each independently of one another represent a C₁ alkyl group.

In addition, it has proven to be advantageous according to the invention if the crosslinked anionic copolymer has structural units of formula (I) in which A represents an NH group and b represents integers from 1 to 3. Advantageous embodiments are therefore characterized in that, in the structural unit of formula (I), A represents NH and b represents integers from 1 to 3.

It is also preferred according to the invention if, in the structural unit of formula (I), the residue X⁺ is selected from particular cations. It is therefore advantageous according to the invention if, in the structural unit of formula (I), X⁺ represents H⁺, NH₄⁺, Na⁺, K⁺, Li⁺, ½Mg²⁺, ½Ca²⁺, or ⅓Al³⁺, in particular NH₄⁺.

Advantageous embodiments of the present invention contain, as thickener, crosslinked anionic copolymers which contain structural units of formulae (Ia) and (IIa). Particularly preferred cosmetic agents according to the invention are therefore characterized in that the crosslinked anionic copolymer contains at least one structural unit of formula (Ia) and at least one structural unit of formula (IIa)

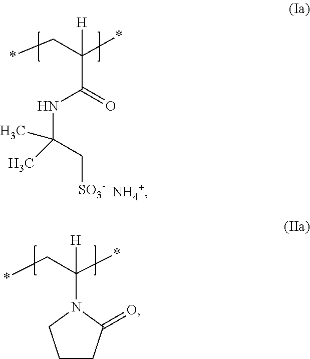

The use of such thickeners leads to a particularly stable thickening of the cosmetic agents according to the invention. Furthermore, such copolymers are sensitive to shearing. The cosmetic agents according to the invention can therefore be packaged in a tube and can be easily removed from the tube under the effect of shear forces and distributed on the keratin fibers, but do not drip from the hair after application to the keratin fibers, that is to say when shear forces are no longer acting on the agent. In this way, an excellent and uniform blonding is achieved even with small amounts of oxidants. In addition, the use of said copolymers also enables a temporary shaping of the keratin fibers. Therefore, with the cosmetic agent according to the invention, both an excellent and uniform blonding and also good long-term hold and good flexibility of the keratin fibers shaped using said agents are achieved.

In the context of the present invention, it has proven to be advantageous if crosslinked anionic copolymers which contain a particular total amount of structural units of formula (I), in particular of the aforementioned formula (Ia), are used as the thickener. It is therefore preferred according to the invention if the crosslinked anionic copolymer contains 49.99 to 98.99 wt %, preferably 69.5 to 97.5, in particular 84.5 to 96.5 wt % structural unit of formula (I), in particular of formula (Ia), in relation to the total weight of the anionic crosslinked copolymer.

Furthermore, it has proven to be advantageous in the context of the present invention if crosslinked anionic polymers which contain a particular total amount of structural units of formula (II), in particular of the aforementioned formula (IIa), are used as the thickener. It is therefore advantageous according to the invention if the crosslinked anionic copolymer contains 1 to 50 wt %, preferably 2 to 30 wt %, in particular 3 to 15 wt % structural unit of formula (II), in particular of formula (IIa), in relation to the total weight of the anionic crosslinked copolymer.

The crosslinking of the anionic copolymer takes place by dispersing or dissolving monomers of the aforementioned formula (I), in particular of formula (Ia), and monomers of the aforementioned formula (II), in particular of formula (IIa), in a protic solvent and adding at least one crosslinking agent having at least two olefinic double bonds. The polymerization can then be started in a manner known per se by adding a radical-forming compound.

According to the invention, use is preferably made of anionic copolymers which have been crosslinked by particular crosslinking agents in particular amounts. Embodiments which are preferred according to the invention are therefore characterized in that the crosslinked anionic copolymer contains 0.01 to 8 wt %, more preferably 0.2 to 3 wt % and particularly preferably 0.5 to 2 wt % crosslinking agent in relation to the total weight of the anionic crosslinked copolymer, said crosslinking agent being selected from the group consisting of allyl acrylate or methacrylate, dipropylene glycol diallyl ether, polyglycol diallyl ether, triethylene glycol divinyl ether, hydroquinone diallyl ether, tetraallyloxyethane or other allyl or vinyl ethers of multifunctional alcohols, tetraethylene glycol diacrylate, triallylamine, trimethylolpropane diallyl ether, methylenebisacrylamide or divinylbenzene, in particular trimethylolpropane diallyl ether.

It is very particularly preferred if the crosslinked anionic copolymer used is a compound which is known under the INCI name Ammonium Acryloyldimethyltaurate/VP Copolymer. This compound has the CAS number 335383-60-3. Such a thickener is commercially available for example under the name “Aristoflex AVC” from the company Clariant.

It is also very particularly preferred if the thickener used is a xanthan compound which has a viscosity of from 800 to 2000 mPas (measured using Brookfield LV at 25° C., 60 rpm, #3 LV spindle) and a mean particle size D90 of less than 180 μm.

Preferred cosmetic agents according to the invention contain the at least one thickener in a total amount of from 0.20 to 7.0 wt %, preferably from 0.40 to 6.0 wt %, more preferably from 0.60 to 5.5 wt %, in particular from 1.0 to 5.0 wt %, in relation to the total weight of the cosmetic agent. If the cosmetic agent according to the invention contains a mixture of thickeners, the amounts specified above relate to the mixture of said thickeners. If, on the other hand, the cosmetic agent contains just one of the aforementioned thickeners, then the amounts specified above relate to said thickener. The use of the aforementioned amounts of thickener results in an excellent thickening of the cosmetic agents according to the invention, but without negatively affecting the removal thereof from a tube or a pot, and in this way enables a uniform distribution and lightening of the keratin fibers. Furthermore, the use of the aforementioned amounts leads to a good hold of the temporarily shaped keratin fibers. In addition, cosmetic agents according to the invention which contain the thickener in the amount ranges specified above can be applied uniformly and are thus easy to handle.

As a second constituent b), the cosmetic agents according to the invention contain at least one oxidant. The latter is able to lighten the hair fibers through oxidative destruction of the hair's own colorant, melanin. It has proven to be advantageous in the context of the present invention if particular oxidants are used. Preferred embodiments of the present invention are therefore characterized in that the oxidant is selected from the group consisting of persulfates, chlorites, hydrogen peroxide, and addition products of hydrogen peroxide with urea, melamine and sodium borate, in particular hydrogen peroxide. For a moderate blonding effect, it is sufficient to use hydrogen peroxide as the oxidant, optionally with the use of ammonia or other alkalizing agents.

The concentration of a hydrogen peroxide solution in the cosmetic agent according to the invention is determined on the one hand by the statutory provisions and on the other hand by the desired effect. Preferably, the cosmetic agent according to the invention contains at least 1.0 wt % hydrogen peroxide (calculated as 100% strength H₂O₂) in relation to the total weight of the cosmetic agent. A uniform and good result of the oxidative treatment is achieved by this minimum concentration. Agents having a lower concentration give non-uniform results.

It is therefore preferred that the oxidant, in particular hydrogen peroxide, is contained in a total amount of from 1.0 to 12 wt %, preferably from 1.3 to 11 wt %, more preferably from 1.7 to 12 wt %, in particular from 2.0 to 10 wt %, in relation to the total weight of the cosmetic composition. If hydrogen peroxide is used as the oxidant, the aforementioned amounts relate to 100% strength H₂O₂. The use of the aforementioned amounts of oxidants, in particular of hydrogen peroxide, leads to a uniform and good lightening of the keratin fibers.

Cosmetic agents according to the invention have a viscosity of from 31,000 to 90,000 mPas. However, it is preferred according to the invention if the agents have the viscosities indicated below. Preferred embodiments of the present invention are therefore characterized in that the cosmetic agent has a viscosity of from 31,000 to 85,000 mPas, preferably from 31,000 to 80,000 mPas, more preferably from 31,000 to 70,000 mPas, in particular from 31,000 to 60,000 mPas (in each case Brookfield RV, spindle T96, 4 rpm). The aforementioned viscosities enable a durable application of the cosmetic agent according to the invention, without dripping, and therefore lead to a uniform lightening. Furthermore, there is no negative effect on the removal of said agent from the packaging, in particular from a tube or a pot.

In the context of the present invention, it has proven to be particularly preferred if the cosmetic agent according to the invention has a particular weight ratio of the at least one thickener to the at least one oxidant. Preferred embodiments of the present invention are therefore characterized in that the cosmetic agent has a weight ratio of the total amount of the at least one thickener to the total amount of the at least one oxidant of from 5:1 to 1:5, preferably from 4:1 to 1:4, more preferably from 3:1 to 1:3, in particular from 2:1 to 1:2. Cosmetic agents which have the aforementioned weight ratios lead to a particularly good and uniform lightening effect and also to excellent hold of the hairstyle.

Blonding processes on keratin fibers usually take place in an alkaline environment. This applies in particular to blonding processes in which the keratin fibers are to be strongly lightened. If the keratin fibers are to be only weakly lightened, it is advantageous to set a low pH so as to be as gentle as possible on the keratin fibers and also on the skin. It is therefore preferred according to the invention if the cosmetic agent has a pH (measured at 22° C.) of from pH 1.0 to pH 7.0, preferably from pH 1.3 to pH 6.8, more preferably from pH 1.6 to pH 6.4, in particular from pH 2.0 to pH 6.0.

By way of example, alkalizing agents can be used to set the preferred pH. Said alkalizing agents can be selected from the group consisting of ammonia, basic amino acids, alkali metal hydroxides, alkanolamines, alkali metal metasilicates, alkali metal phosphates and alkali metal hydrogen phosphates. The alkali metal ions used are preferably lithium, sodium, potassium, in particular sodium or potassium.

The basic amino acids which can be used as alkalizing agents are preferably selected from the group consisting of L-arginine, D-arginine, D,L-arginine, L-lysine, D-lysine, D,L-lysine, particularly preferably L-arginine, D-arginine, D,L-arginine used as an alkalizing agent in the context of the invention.

The alkali metal hydroxides which can be used as alkalizing agents are preferably selected from the group consisting of sodium hydroxide and potassium hydroxide.

The alkanolamines which can be used as alkalizing agents are preferably selected from primary amines having a C₂-C₆ alkyl main body which carries at least one hydroxyl group. Particularly preferred alkanolamines are selected from the group formed of 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Alkanolamines which are very particularly preferred according to the invention are selected from the group consisting of 2-aminoethan-1-ol, 2-amino-2-methylpropan-1-ol and 2-amino-2-methylpropane-1,3-diol.

To stabilize the at least one oxidant, in particular the hydrogen peroxide, the cosmetic agent according to the invention may additionally contain particular acids. It is therefore advantageous in the context of the present invention if the cosmetic agent additionally contains at least one acid selected from the group consisting of dipicolinic acid, edible acids, such as for example citric acid, acetic acid, malic acid, lactic acid and tartaric acid, dilute mineral acids, such as hydrochloric acid, phosphoric acid, pyrophosphoric acid and salts thereof, 1-hydroxyethane-1,1-diphosphonic acid, sulfuric acid, ethylenediaminetetraacetic acid and salts thereof, and mixtures thereof, in particular dipicolinic acid and/or sodium pyrophosphate and/or 1-hydroxyethane-1,1-diphosphonic acid and/or ethylenediaminetetraacetic acid and salts thereof. The use of acids in the form of dipicolinic acid and/or sodium pyrophosphate has proven to be particularly advantageous with regard to stabilizing the hydrogen peroxide. Furthermore, by adding the complexing agents 1-hydroxyethane-1,1-diphosphonic acid and/or ethylenediaminetetraacetic acid, it is possible to avoid an undesired color change caused by the presence of heavy metal ions during the lightening. Particularly preferred cosmetic agents therefore contain a mixture of dipicolinic acid and sodium pyrophosphate as well as at least one of the complexing agents 1-hydroxyethane-1,1-diphosphonic acid and/or ethylenediaminetetraacetic acid.

To ensure sufficient stabilization of the oxidant, in particular of the hydrogen peroxide, and to avoid undesired color changes, it is advantageous in this connection if the aforementioned at least one acid is used in particular total amounts. It is therefore preferred in this connection if the at least one acid, in particular dipicolinic acid and/or sodium pyrophosphate and/or 1-hydroxyethane-1,1-diphosphonic acid and/or ethylenediaminetetraacetic acid and salts thereof, is contained in a total amount of from 0.1 to 3.0 wt %, preferably from 0.5 to 2.5 wt %, more preferably from 0.8 to 2.0 wt %, in particular from 0.9 to 1.5 wt %, in relation to the total weight of the composition. If just one acid is additionally used, the amounts specified above relate to said acid. If, on the other hand, the cosmetic agents according to the invention contain a mixture of the aforementioned acids, then said specified amounts relate to the mixture of said acids.

It may be provided according to the invention that the cosmetic agent additionally contains further ingredients besides the substances mentioned above. In this connection, it is advantageous if the cosmetic agents according to the invention contain at least one nonionic surfactant in order to avoid any precipitation or settling of the constituents. It is therefore preferred in the context of the present invention if the cosmetic agent additionally contains at least one nonionic surfactant selected from the group consisting of alkyl polyglucosides, sorbitan esters with 1 to 10 mol ethylene oxide per mole of sorbitan esters, C₈-C₃₀ carboxylic acid monoethanolamide, ethoxylated C₈-C₃₀ alcohols with 4 to 20 mol ethylene oxide per mole of C₈-C₃₀ alcohol, addition products of 20 to 60 mol ethylene oxide with castor oil and hydrogenated castor oil, and mixtures thereof, in particular addition products of 20 to 60 mol ethylene oxide with hydrogenated castor oil.

In this connection, it is advantageous if the at least one nonionic surfactant, addition products of 20 to 60 mol ethylene oxide with hydrogenated castor oil, is contained in a total amount of from 0.1 to 3.0 wt %, preferably from 0.2 to 2.0 wt %, more preferably from 0.3 to 1.5 wt %, in particular from 0.4 to 1.0 wt %, in relation to the total weight of the composition. The use of the aforementioned total amounts of nonionic surfactant leads to a good storage stability of the cosmetic agents according to the invention since settling, phase separation or precipitation of constituents during storage is avoided.

In addition, the cosmetic agent according to the invention may also contain care substances. These include for example fatty substances, cosmetic oil bodies, plant extracts, protein hydrolysates, vitamins, provitamins, vitamin precursors, polysiloxanes, cationic polymers, amphoteric polymers, and mixtures thereof. The combination of panthenol (provitamin B5) and protein hydrolysates has proven to be a particularly preferred combination of care substances.

The cosmetic agent according to the invention may additionally contain preferably nonionic polymers, perfume, opacifiers and/or colorants for coloring the liquid.

The cosmetic agent according to the invention is preferably packaged in a tube or a pot and, after removal of a particular amount, is applied to the keratin fibers.

Particularly preferred embodiments AF 1 to AF 80 of the cosmetic agents according to the invention are listed in the tables below (unless indicated otherwise, all figures are specified in wt %). Embodiments AF 1 to AF 80 preferably have a viscosity of from 31,000 to 60,000 mPas (in each case Brookfield RV, spindle T96, 4 rpm) and a pH of from 2.0 to pH 6.0 (measured at 22° C.).

| | AF 1 | AF 2 | AF 3 | AF 4 |
|---|---|---|---|---|
| Xanthan | 0.20- | 0.40-6.0 | 0.60-5.5 | 1.0-5.0 |
| | 7.0 | | | |
| Oxidant | 1.0-12 | 1.3-11 | 1.7-12 | 2.0-10 |
| Cosmetic carrier ¹⁾ | ad 100 | ad 100 | ad 100 | ad 100 |
| | AF 5 | AF 6 | AF 7 | AF 8 |
| Crosslinked anionic copolymer ²⁾ | 0.20- | 0.40-6.0 | 0.60-5.5 | 1.0-5.0 |
| | 7.0 | | | |
| Oxidant | 1.0-12 | 1.3-11 | 1.7-12 | 2.0-10 |
| Cosmetic carrier ¹⁾ | ad 100 | ad 100 | ad 100 | ad 100 |
| | AF 9 | AF 10 | AF 11 | AF 12 |
| Thickener ³⁾ | 0.20- | 0.40-6.0 | 0.60-5.5 | 1.0-5.0 |
| | 7.0 | | | |
| Oxidant | 1.0-12 | 1.3-11 | 1.7-12 | 2.0-10 |
| Cosmetic carrier ¹⁾ | ad 100 | ad 100 | ad 100 | ad 100 |
| | AF 13 | AF 14 | AF 15 | AF 16 |
| Xanthan | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Cosmetic carrier ¹⁾ | ad 100 | ad 100 | ad 100 | ad 100 |
| | AF 17 | AF 18 | AF 19 | AF 20 |
| Crosslinked anionic copolymer ²⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Cosmetic carrier ¹⁾ | ad 100 | ad 100 | ad 100 | ad 100 |
| | AF 21 | AF 22 | AF 23 | AF 24 |
| Thickener ³⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Cosmetic carrier ¹⁾ | ad 100 | ad 100 | ad 100 | ad 100 |
| | AF 25 | AF 26 | AF 27 | AF 28 |
| Crosslinked anionic copolymer ⁵⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Cosmetic carrier ¹⁾ | ad 100 | ad 100 | ad 100 | ad 100 |
| | AF 29 | AF 30 | AF 31 | AF 32 |
| Thickener ⁶⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Cosmetic carrier ¹⁾ | ad 100 | ad 100 | ad 100 | ad 100 |
| | AF 33 | AF 34 | AF 35 | AF 36 |
| Crosslinked anionic copolymer ⁷⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Cosmetic carrier ¹⁾ | ad 100 | ad 100 | ad 100 | ad 100 |
| | AF 37 | AF 38 | AF 39 | AF 40 |
| Thickener ⁸⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Cosmetic carrier ¹⁾ | ad 100 | ad 100 | ad 100 | ad 100 |
| | AF 41 | AF 42 | AF 43 | AF 44 |
| Xanthan | 0.20- | 0.40-6.0 | 0.60-5.5 | 1.0-5.0 |
| | 7.0 | | | |
| Oxidant | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |
| | AF 45 | AF 46 | AF 47 | AF 48 |
| Crosslinked anionic copolymer ²⁾ | 0.20- | 0.40-6.0 | 0.60-5.5 | 1.0-5.0 |
| | 7.0 | | | |
| Oxidant | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |
| | AF 49 | AF 50 | AF 51 | AF 52 |
| Thickener ³⁾ | 0.20- | 0.40-6.0 | 0.60-5.5 | 1.0-5.0 |
| | 7.0 | | | |
| Oxidant | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |
| | AF 53 | AF 54 | AF 55 | AF 56 |
| Xanthan | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |
| | AF 57 | AF 58 | AF 59 | AF 60 |
| Crosslinked anionic copolymer ²⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |
| | AF 61 | AF 62 | AF 63 | AF 64 |
| Thickener ³⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |
| | A 65 | AF 66 | AF 67 | AF 68 |
| Crosslinked anionic copolymer ⁵⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |
| | AF 69 | A 70 | AF 71 | AF 72 |
| Thickener ⁶⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |
| | AF 73 | AF 74 | AF 75 | AF 76 |
| Crosslinked anionic copolymer ⁷⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |
| | AF 77 | AF 78 | AF 79 | AF 80 |
| Thickener ⁸⁾ | 0.20- | 0.40-6.0 | 0.60-5.5 | 1.0-5.0 |
| | 7.0 | | | |
| Hydrogen peroxide ⁴⁾ | 1.0-12 | 1.3-11 | 1.7-12 | 2.0-10 |
| Acid ⁹⁾ | 0.1-3.0 | 0.5-2.5 | 0.8-2.0 | 0.9-1.5 |
| Cosmetic carrier ¹⁾ | ad 100 | ad 100 | ad 100 | ad 100 |

¹⁾ selected from aqueous and aqueous-alcoholic carriers
²⁾ including at least one above-described structural unit of formula (I) and at least one above-described structural unit of formula (II)
³⁾ mixture of xanthan and at least one crosslinked anionic copolymer, including at least one above-described structural unit of formula (I) and at least one above-described structural unit of formula (II)
⁴⁾ specified amounts relate to 100% strength hydrogen peroxide
⁵⁾ includes at least one above-described structural unit of formula (Ia) and at least one above-described structural unit of formula (IIa)
⁶⁾ mixture of xanthan and at least one crosslinked anionic copolymer, including at least one above-described structural unit of formula (Ia) and at least one above-described structural unit of formula (IIa)
7) including 84.5 to 96.5 wt % structural unit of formula (Ia) and 3 to 15 wt % structural unit of formula (IIa), in each case in relation to the total weight of the anionic crosslinked copolymer
⁸⁾ mixture of xanthan and at least one crosslinked anionic copolymer, including 84.5 to 96.5 wt % structural unit of formula (Ia) and 3 to 15 wt % structural unit of formula (IIa), in each case in relation to the total weight of the anionic crosslinked copolymer
⁹⁾ selected from dipicolinic acid and/or sodium pyrophosphate and/or 1-hydroxyethane-1,1-diphosphonic acid and/or ethylenediaminetetraacetic acid

Due to the sensitivity of the thickeners to shear, the embodiments listed above can be easily removed from the packaging, in particular from a tube or a pot, but after application have a viscosity that is optimal in terms of the ease of application and dosing, so that dripping after application can be avoided. This leads to a uniform and excellent lightening of the keratin fibers, even when small amounts of oxidants are used. In addition, the use of the aforementioned embodiments AF 1 to AF 80 leads to an excellent hairstyle hold and also to good flexibility of the keratin fibers temporarily shaped using said agents.

A second subject matter of the present invention is a method for lightening and temporarily shaping keratin fibers, wherein a cosmetic agent according to the invention is applied to the keratin fibers and the latter are then shaped as desired.

With regard to further preferred embodiments of the method according to the invention, what has been stated in relation to the cosmetic agents according to the invention applies, mutatis mutandis.

Finally, a further subject matter of the present invention is the use of a cosmetic agent according to the invention for lightening and temporarily shaping keratin fibers.

With regard to further preferred embodiments of the use according to the invention, what has been stated in relation to the cosmetic agents according to the invention and in relation to the method according to the invention applies, mutatis mutandis.

The following examples explain the present invention, but without limiting the invention thereto:

### Examples

Unless indicated otherwise, all specified amounts are parts by weight of active substance. The following recipes were prepared using known production methods. The crosslinked anionic copolymer used is preferably a copolymer which includes 84.5 to 96.5 wt % structural units of formula (Ia) and 3 to 15 wt % structural units of formula (IIa), in each case in relation to the total weight of the copolymer, with the aforementioned meanings of the residues in said formulae. The oxidant used is preferably hydrogen peroxide.

| | 1 | 2 | 3 | 4 | 5 |
|---|---|---|---|---|---|
| Xanthan gum | 5.0 | 3.0 | 2.0 | 4.0 | 1.0 |
| Oxidant | 3.8 | 4.0 | 1.5 | 2.5 | 3.0 |
| Ammonia (25%) | 0.6 | 0.6 | 0.6 | 0.6 | 0.6 |
| Dipicolinic acid | 0.1 | 0.1 | 0.1 | 0.1 | 0.1 |
| Disodium dihydrogen diphosphate | 0.03 | 0.03 | 0.03 | 0.03 | 0.03 |
| 1-Hydroxyethane-(1,1-diphosphonic acid) | 1.5 | 1.5 | 1.5 | 1.5 | 1.5 |
| (60%) | | | | | |
| D-Panthenol (75%) | 0.32 | 0.32 | 0.32 | 0.32 | 0.32 |
| Ethoxylated hydrogenated castor oil (40 | 0.50 | 0.50 | 0.50 | 0.50 | 0.50 |
| EO) | | | | | |
| Orange oil | 0.08 | 0.08 | 0.08 | 0.08 | 0.08 |
| Crodarom Pearl | 1.0 | 1.0 | 1.0 | 1.0 | 1.0 |
| 1,2-Propanediol | 10 | 10 | 10 | 10 | 10 |
| Perfume | 0.1 | 0.1 | 0.1 | 0.1 | 0.1 |
| Water | ad | ad | ad | ad | ad |
| | 100 | 100 | 100 | 100 | 100 |
| | 6 | 7 | 8 | 9 | 10 |
| Crosslinked anionic copolymer | 5.0 | 3.0 | 2.0 | 4.0 | 1.0 |
| Oxidant | 3.8 | 4.0 | 1.5 | 2.5 | 3.0 |
| Ammonia (25%) | 0.6 | 0.6 | 0.6 | 0.6 | 0.6 |
| Dipicolinic acid | 0.1 | 0.1 | 0.1 | 0.1 | 0.1 |
| Disodium dihydrogen diphosphate | 0.03 | 0.03 | 0.03 | 0.03 | 0.03 |
| 1-Hydroxyethane-(1,1-diphosphonic acid) | 1.5 | 1.5 | 1.5 | 1.5 | 1.5 |
| (60%) | | | | | |
| D-Panthenol (75%) | 0.32 | 0.32 | 0.32 | 0.32 | 0.32 |
| Ethoxylated hydrogenated castor oil (40 | 0.50 | 0.50 | 0.50 | 0.50 | 0.50 |
| EO) | | | | | |
| Orange oil | 0.08 | 0.08 | 0.08 | 0.08 | 0.08 |
| Crodarom Pearl | 1.0 | 1.0 | 1.0 | 1.0 | 1.0 |
| 1,2-Propanediol | 10 | 10 | 10 | 10 | 10 |
| Perfume | 0.1 | 0.1 | 0.1 | 0.1 | 0.1 |
| Water | ad | ad | ad | ad | ad |
| | 100 | 100 | 100 | 100 | 100 |

Cosmetic agents 1 to 10 according to the invention have a viscosity of from 31,000 to 90,000 mPas (in each case measuring using Brookfield RV, spindle T96, 4 rpm) and were able to be finely and homogeneously applied to human hair. In particular, said agents were able to be removed very easily from a tube or a pot. After application to the hair, the viscosity of said agents significantly increased after a very short period of time (less than 5 seconds), so that said agents did not drip from the hair. A very homogeneous lightening of the hair and also a good hairstyle hold of the keratin fibers shaped using said agents were achieved.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent for lightening and temporarily shaping keratin fibers, containing, in a cosmetically acceptable carrier,
a) at least one thickener, selected from xanthan gum and/or crosslinked anionic copolymers, including at least one structural unit of formula (I) and at least one structural unit of formula (II) 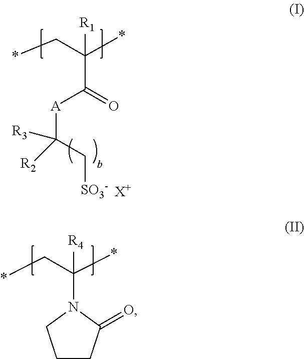
in which
R₁ and R₄ each independently of one another represent hydrogen or a C₁₋₄ alkyl group,
R₂ and R₃ each independently of one another represent a C₁₋₄ alkyl group,
A represents O, NH, or S,
b represents integers from 1 to 5,
X⁺ represents a physiologically acceptable cation, and
b) at least one oxidant,
wherein the cosmetic agent has a viscosity of from 31,000 to 90,000 mPas (Brookfield RV, spindle T96, 4 rpm).

2. The cosmetic agent according to claim 1, wherein the residue R₁ in the structural unit of formula (I) and the residue R₄ in the structural unit of formula (II) each represent hydrogen.

3. The cosmetic agent according to claim 1, wherein in the structural unit of formula (I), the residues R₂ and R₃ each independently of one another represent a C₁ alkyl group.

4. The cosmetic agent according to claim 1, wherein the crosslinked anionic copolymer contains at least one structural unit of formula (Ia) and at least one structural unit of formula (IIa)

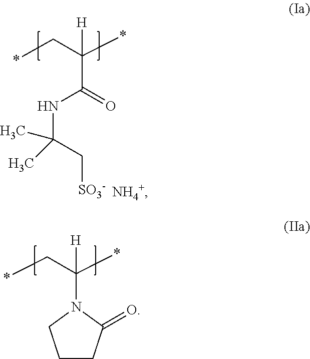

5. The cosmetic agent according to claim 1, wherein the thickener is contained in a total amount of from 0.20 to 7.0 wt % in relation to the total weight of the cosmetic agent.

6. The cosmetic agent according to claim 1, wherein the oxidant is selected from the group consisting of persulfates, chlorites, hydrogen peroxide, and addition products of hydrogen peroxide with urea, melamine, and sodium borate.

7. The cosmetic agent according to claim 1, wherein the oxidant, in particular hydrogen peroxide, is contained in a total amount of from 1.0 to 12 wt % in relation to the total weight of the cosmetic composition.

8. The cosmetic agent according to claim 1, wherein the cosmetic agent has a viscosity of from 31,000 to 85,000 mPas measured with a Brookfield RV, spindle T96, 4 rpm,

9. A method for lightening and temporarily shaping keratin fibers, wherein a cosmetic agent according to claim 1 is applied to the keratin fibers and the latter are then shaped as desired.
